(19) United States
(12) Patent Application Publication
(10) Pub. No.: US 2025/0339273 A1
(43) Pub. Date: Nov. 6, 2025

(54) HEART CHAMBER PRESSURE MODULATION

(71) Applicant: EDWARDS LIFESCIENCES CORPORATION, Irvine, CA (US)

(72) Inventors: Zachary Christopher Berwick, Tustin, CA (US); Allen Jeong Keel, Irvine, CA (US); Yaeer E. Lev, Torrance, CA (US)

(21) Appl. No.: 19/265,730

(22) Filed: Jul. 10, 2025

Related U.S. Application Data
(63) Continuation of application No. PCT/US2024/011528, filed on Jan. 13, 2024.
(60) Provisional application No. 63/481,111, filed on Jan. 23, 2023.

(51) Int. Cl. A61F 2/24; A61F 2/48

(52) U.S. Cl. CPC A61F 2/2487; A61F 2/482; A61F 2220/0016; A61F 2230/0091

(57) ABSTRACT

A method for assisting a heart ventricle can comprise providing a longitudinally expandable member that is reversibly expandable along a longitudinal dimension, wherein the longitudinally expandable member can include adjacent abutting portions meeting at respective bends that form acute angles along the longitudinal dimension. The method can include securing a first end portion of the longitudinally expandable member to a first wall portion of the heart ventricle via a first anchor engagement feature associated with the first end portion. The method can include securing a second end portion of the longitudinally expandable member to a second wall portion of the heart ventricle via a second anchor engagement feature associated with the second end portion, the plurality of bends between the first and second end portions allowing augmenting expansion of the heart ventricle using spring force of the longitudinally expandable member to thereby improve heart function.

### CROSS REFERENCE TO RELATED APPLICATION(S)

This application is a continuation of International Patent Application No. PCT/US2024/011528, filed Jan. 13, 2024, which claims the benefit of U.S. Provisional Patent Application No. 63/481, 111, filed on Jan. 23, 2023, the complete disclosures of each of which are hereby incorporated by reference in their entireties for all purposes.

### BACKGROUND

Atrial fibrillation and heart failure can be co-existing conditions. Heart failure can contribute to atrial fibrillation, and, conversely, atrial fibrillation can contribute to heart failure. For example, elevated filling pressures of a left heart ventricle, altered handling by the heart of intracellular calcium, and/or autonomic and neuroendocrine dysfunction resulting from heart failure can contribute to onset of left atrial fibrillation. Atrial fibrillation can produce exaggerated heart rate responses, such as when exercising, and reduced ventricular filling times, which can contribute to heart failure.

### SUMMARY

Described herein are methods and devices relating to modulating pressure of a heart chamber, including a heart atrium, such as the left atrium. In some instances, methods and/or devices described herein can be configured to augment expansion and/or untwisting of a heart chamber, such as a heart ventricle, including the left ventricle, during ventricular diastole. Augmenting expansion and/or untwisting of the left ventricle can provide an increased volume for the ventricle during ventricular diastole, improving emptying of the left atrium into the left ventricle, thereby facilitating modulation of the left atrium. In some instances, a medical implant assembly can comprise a longitudinally expandable member configured to be reversibly expandable along a longitudinal dimension of the longitudinally expandable member. The longitudinally expandable member can supplement extension and/or lengthening of one or more ventricular myocardial muscle fibers, including for example, subendocardial longitudinal fibers of a left ventricle. In some instances, a medical implant device can comprise a circumferentially disposed portion configured to be reversibly expandable radially. The circumferentially disposed portion can be configured to supplement radial expansion of one or more ventricular myocardial muscle fibers, including for example, the subendocardial circumferential fibers of a left ventricle. In some instances, a medical implant device can comprise a circumferentially disposed portion and a rod coupled to a central portion of the circumferentially disposed portion configured to be anchored to an apical region of the heart. The circumferentially disposed portion can be configured to supplement radial expansion of one or more ventricular myocardial muscle fibers, including for example, the subendocardial circumferential fibers of a left ventricle. The rod can be configured to supplement untwisting in the apical region of the heart ventricle.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

For purposes of summarizing the disclosure, certain aspects, advantages, and novel features have been described herein. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements. However, it should be understood that the use of similar reference numbers in connection with multiple drawings does not necessarily imply similarity between respective examples associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some examples or configurations.

FIG. 1 provides a cut-away view of a human heart.

FIG. 2 provides a cut-away view of a human heart and a perspective view of a medical implant assembly comprising a longitudinally expandable member deployed to a ventricular heart wall of the heart, in accordance with one or more examples.

FIGS. 3A and 3B provide perspective views of the medical implant assembly described with reference to FIG. 2 in a compressed state and an expanded state, respectively, in accordance with one or more examples.

FIG. 4 provides a cut-away view of a human heart, and a side view of a medical device assembly that includes a longitudinally expandable member comprising an electrically activated polymer, in accordance with one or more examples.

FIGS. 5 provides a perspective view of a delivery assembly configured to deliver a medical device assembly described herein, in accordance with one or more examples.

FIG. 6 provides a perspective view of a medical implant device comprising a circumferentially disposed portion configured to be reversibly expandable radially, in accordance with one or more examples.

FIG. 7 provides a perspective view of another medical implant device comprising a circumferentially disposed portion configured to be reversibly expandable radially, in accordance with one or more examples.

FIG. 8 provides a perspective view of a medical implant device that includes a circumferentially disposed portion comprising a plurality of radially extending spokes, in accordance with one or more examples.

FIG. 9 provides a cut-away view of a human heart and a perspective view of a medical implant device deployed into a heart ventricle of the heart, where the medical implant device comprises a circumferentially disposed portion and a rod coupled to the circumferentially disposed portion, in accordance with one or more examples.

FIG. 10A shows the medical implant device described with reference to FIG. 9 disposed within a heart ventricle that is in a diastole phase. FIG. 10B shows the medical implant device described with reference to FIG. 9 disposed within the heart ventricle while the ventricle begins to contract during the systole phase, and FIG. 10C shows the medical implant device disposed within the heart ventricle while the ventricle contracts further and assumes a fully compressed state during the systole phase.

### DETAILED DESCRIPTION

The headings provided herein are for convenience only and do not necessarily affect the scope or meaning of the claimed invention.

Although certain preferred examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain standard anatomical terms of location are used herein to refer to the anatomy of animals, and namely humans, with respect to the preferred examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

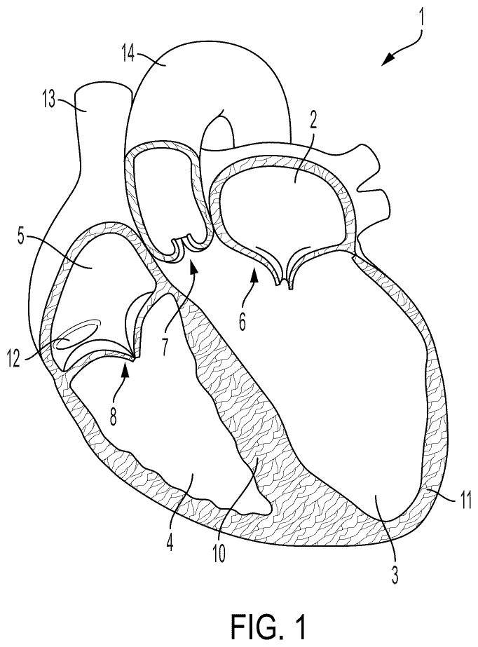

FIG. 1 shows certain anatomical features of human vasculature, including various features of a human heart 1. The heart 1 includes four chambers, namely the left atrium 2, the left ventricle 3, the right ventricle 4, and the right atrium 5. A wall of muscle, referred to as the septum 10, separates the left atrium 2 and right atrium 5, and the left ventricle 3 and right ventricle 4. Blood flow through the heart 1 is at least partially controlled by four valves, the mitral valve 6, aortic valve 7, tricuspid valve 8, and pulmonary valve (not shown). The mitral valve 6 separates the left atrium 2 and the left ventricle 3 and controls blood flow therebetween. The aortic valve 7 separates and controls blood flow between the left ventricle 3 and the aorta 14. The tricuspid valve 8 separates the right atrium 5 and the right ventricle 4 and controls blood flow therebetween. The pulmonary valve separates the right ventricle 4 and the pulmonary trunk or artery (not shown), controlling blood flow therebetween.

In a healthy heart, the heart valves can properly open and close in response to a pressure gradient present during various stages of the cardiac cycle (e.g., relaxation and contraction) to at least partially control the flow of blood to a respective region of the heart and/or to blood vessels. Deoxygenated blood arriving from the rest of the body generally flows into the right side of the heart for transport to the lungs, and oxygenated blood from the lungs generally flows into the left side of the heart for transport to the rest of the body. During ventricular diastole, deoxygenated blood arrive in the right atrium 5 from the inferior vena cava 12 and superior vena cava 13 to flow into the right ventricle 4, and oxygenated blood arrive in the left atrium 2 from the pulmonary veins to flow into the left ventricle 3. During ventricular systole, deoxygenated blood from the right ventricle 4 can flow into the pulmonary trunk for transport to the lungs (e.g., via the left and right pulmonary arteries), and oxygenated blood can flow from the left ventricle 3 to the aorta 14 for transport to the rest of the body.

Heart failure and atrial fibrillation can be co-existing conditions, including in heart failure with preserved ejection fraction (HFpEF). During atrial fibrillation, the atrium may not sufficiently contract, which can result in an increase in residual volume at the end of atrial systolic phase. Inflow during the filling period of the atrium can subsequently elevate the pressure, reduce compliance, and/or lower reservoir strain of the atrium. Progression in atrial fibrillation can be reflected by atrial fibrillation burden, which can be reflected by the longest atrial fibrillation episode, the number of atrial fibrillation episodes, and/or the percentage of time in atrial fibrillation during a monitoring period. For example, over time, the sustained increase in pressure can progressively dilate the left atrium, which can further increase the frequency of atrial fibrillation. Paroxysmal atrial fibrillation can transition to permanent atrial fibrillation. Patients suffering from permanent atrial fibrillation undergoing atrial ablation procedures can experience a low success rate.

Modification of ventricular pressure can facilitate modulation of atrial pressure. For example, a modulator of left atrial pressure can be the left ventricle. Elevated left ventricular pressure can increase the resistance to emptying of the left atrium, increasing the left atrial pressure. The left ventricular myocardium can comprise three layers of muscle, each extending along a different axis. Each of the layers can contract and expand along a corresponding axis. The muscle layers can work in concert to compress the left ventricle for pumping blood out of the left ventricle and to relax the left ventricle during filling of the left ventricle. For example, the inner layer of the subendocardium can be oriented longitudinally. The middle layer can be oriented circumferentially. The outer subepicardial layer can be oriented obliquely. These layers of the myocardium can contract along their respective directions to produce a wringing motion, for example the wringing motion being apparent at the apex of the heart. In some cases, during heart failure, the inner layer can fail first. Other layers of the myocardium can compensate for the inner layer, including for example the outer layer of the myocardium. The outer layer can eventually fail as well. Failure of one or more of the myocardium layers can lead to dilation of the left ventricle, and reduced ejection fraction heart failure. Dilation of the left ventricle and/or reduced ejection fraction heart failure can impede emptying of the left atrium into the left ventricle, increasing left atrial pressure.

Described herein are methods, devices and/or systems configured to provide improved emptying of a heart chamber, such as a heart atrium, including the left atrium. For example, improved emptying of the left atrium can facilitate reduced progressive increases in left pressure-volume, thereby reducing or preventing dilation. The devices and/or assemblies can be configured to enhance relaxation of one or more layers of the layers of the left ventricular myocardium. In some instances, the devices and/or assemblies can be configured to augment lengthening and/or radial expansion of a layer of the ventricular myocardium. In some instances, the device and/or assembly can be configured to augment untwisting of the ventricle. For example, a device and/or assembly can be configured to augment lengthening and/or radial expansion of muscle fibers, and/or supplement the untwisting motion, during diastole phase of the heart ventricle, including for example during early or late diastole. Increase in left atrial reservoir loading during atrial fibrillation can be reduced by augmenting the left atrial conduit function, which can offset the volume of blood that would have typically been ejected during active left atrial pump function but is lost due to atrial fibrillation. Enhanced lengthening and/or radial expansion of the muscle fibers, and/or improved untwisting of the heart ventricle, can provide increased rate and/or duration of diastolic filling of the heart ventricle, improving emptying of the heart atrium into the heart ventricle. Forces that contribute to progressive dilation of the left atrium can be reduced and thereby reducing atrial fibrillation burden, reducing or preventing the transition into permanent atrial fibrillation.

In some instances, a medical implant assembly can comprise a longitudinally expandable member configured to be reversibly expandable along a longitudinal dimension of the longitudinally expandable member. The longitudinally expandable member can supplement extension and/or lengthening of one or more ventricular myocardial muscle fibers, including for example, subendocardial longitudinal fibers of a left ventricle. In some instances, a medical implant device can comprise a circumferentially disposed portion configured to be reversibly expandable radially. The circumferentially disposed portion can be configured to supplement radial expansion of one or more ventricular myocardial muscle fibers, including for example, the subendocardial circumferential fibers of a left ventricle. In some instances, a medical implant device can comprise a circumferentially disposed portion and a rod coupled to a central portion of the circumferentially disposed portion configured to be anchored to an apical region of the heart. The circumferentially disposed portion can be configured to supplement radial expansion of one or more ventricular myocardial muscle fibers, including for example, the subendocardial circumferential fibers of a left ventricle. The rod can be configured to supplement untwisting in the apical region of the heart ventricle.

Deployment of one or more devices and/or assemblies described herein into a heart chamber can comprise access via a transseptal approach, including an atrial transseptal and/or a ventricular transseptal approach. It will be understood that one or more of the longitudinally expandable members, the circumferentially disposed portion and/or the circumferentially disposed portion and rod can be used in combination. For example, one or more of the longitudinally expandable members, the circumferentially disposed portion and/or the circumferentially disposed portion and rod can be deployed into a heart ventricle.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The term “associated with” is used herein according to its broad and ordinary meaning. For example, where a first feature, clement, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

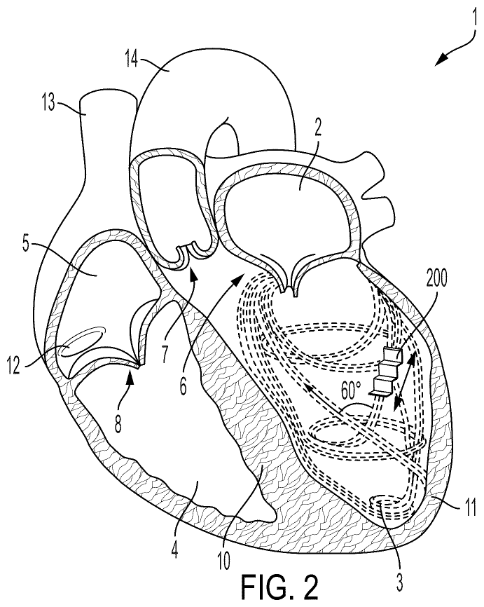

FIG. 2 shows a cut-away view of the heart 1 and an example of a medical implant assembly 200 coupled to a ventricular heart wall 11 of the heart 1. The medical implant assembly 200 can be secured to a ventricular heart wall surface oriented toward a heart ventricle. FIG. 2 shows the medical implant assembly 200 positioned within the left heart ventricle 3. In some instances, the medical implant assembly 200 can be positioned over an inner subendocardial fiber of the left heart ventricle 3. The medical implant assembly 200 can be positioned along an inner subendocardial fiber axis. For example, an orientation of the medical implant assembly 200 can be aligned with that of an axis of the subendocardial longitudinal fibers. In some instances, the medical implant assembly 200 can be secured to a portion of the ventricular heart wall surface aligned with the axis of the subendocardial longitudinal fibers of the left heart ventricle 3. As described in further detail herein, the medical implant assembly 200 can comprise a longitudinally expandable member 202. For example, the longitudinally expandable member 202 can be configured to be secured to the ventricular heart wall surface along an inner subendocardial fiber axis. An orientation of the longitudinally expandable member 202 can be aligned with that of the axis of the subendocardial longitudinal fibers of the left heart ventricle 3.

FIG. 2 shows one medical implant assembly 200 deployed into the left heart ventricle 3. In some instances, a plurality of medical implant assemblies 200 can be deployed into the left heart ventricle 3. The medical implant assemblies 200 can be coupled to respective portions of the ventricular heart wall 11 along the axis of the subendocardial longitudinal fibers. For example, a plurality of longitudinally expandable members 202 can be configured to be secured to respective positions on the ventricular heart wall surface. The plurality of longitudinally expandable members 202 can be arranged along a dimension aligned with the inner subendocardial fiber axis. Each of the plurality of medical implant assemblies 200 may or may not be coupled to an adjacent medical implant assembly 200. For example, each of the plurality of medical implant assemblies 200 can be separate from one another, or the plurality of medical implant assemblies 200 can form a continuous unit.

Layers of the ventricular myocardium, such as left ventricular myocardium, can be in a relaxed state during the early phase of ventricular diastole. For example, elongation of subendocardial and subepicardial fibers can promote an untwisting of the apex. Impairment of the subendocardial fibers can result in an imbalance in the contraction and/or relaxation of the muscle fibers. For example, exaggerated contraction of the subepicardial layer can occur due to dysfunction of the subendocardial fibers, contributing to exaggerated circumferential deformation of the heart ventricle. In some instances, the medical implant assembly 200 can be deployed to be over and aligned with the subendocardial fibers so as to provide at least some compensation for the dysfunction of the subendocardial fibers. For example, a longitudinal axis of the longitudinally expandable member 202 can be aligned with the axis of the subendocardial longitudinal fibers, such as extending along a direction that the subendocardial fibers extend. FIG. 2 shows in dashed lines examples of subendocardial longitudinal fibers of the heart 1. The medical implant assembly 200 is shown as being positioned over subendocardial fibers and to extend along the same direction as that of the subendocardial fibers. In some instances, the medical implant assembly 200 can be configured to provide supplemental extension and/or lengthening of the subendocardial longitudinal fibers. Supplementing the extension and/or lengthening of the subendocardial longitudinal fibers, such as during relaxation of the subendocardial longitudinal fibers, can increase a volume of the ventricle during diastole. For example, supplementing the extension and/or lengthening of the subendocardial longitudinal fibers can improve emptying of the left atrium into the left ventricle during ventricular diastole. Extension of subendocardial longitudinal fibers can be out of plane with contraction of other muscle fibers, which are oblique or tangential, thereby reducing or preventing the impact on systolic wall stress while promoting the relaxation at cither early or late diastole.

In some instances, the one or more medical implant assemblies can be arranged along a dimension of the heart ventricle that is between about 25% to about 75% of a longitudinal dimension, such as a length, of the heart ventricle. In some instances, the one or more medical implant assemblies can be arranged along a dimension of the heart ventricle that is between about 3 centimeters (cm) and about 10 centimeters (cm). In some instances, one medical implant assembly having the desired length can be used. For example, a single medical implant assembly can have a length of about 25% to about 75% of a length of the heart ventricle. In some instances, the medical implant assembly can have a length of about 3 centimeters (cm) to about 10 centimeters (cm). In some instances, a plurality medical implant assemblies can be arranged along a portion of the heart ventricle wall that is about 25% to about 75% of a length of the heart ventricle. In some instances, the plurality of medical implant assemblies can be arranged along a portion of the heart ventricle wall that is about 3 centimeters (cm) to about 10 centimeters (cm).

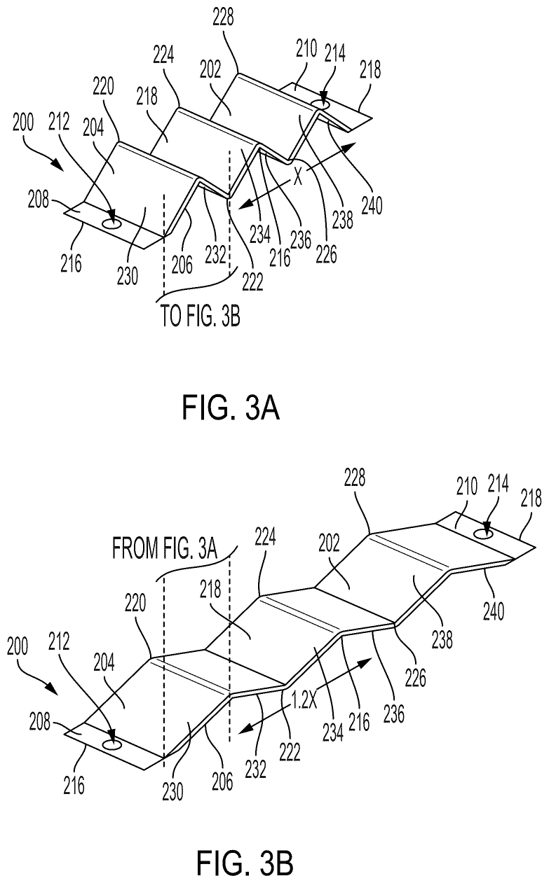

As described herein, the medical implant assembly 200 can comprise the longitudinally expandable member 202. FIG. 3A is a perspective view of the longitudinally expandable member 202 in a compressed state and FIG. 3B is a perspective view of the expandable member 202 in an expanded state. The longitudinally expandable member 202 can comprise an elongate portion configured to be reversibly expandable along a longitudinal dimension of the longitudinally expandable member 202. In some instances, the longitudinally expandable member 202 can be configured to expand as the heart ventricle to which the longitudinally expandable member 202 is deployed expands and/or relaxes during diastole. For example, the longitudinally expandable member 202 can be configured to expand during diastole. The longitudinally expandable member 202 can be configured to assume the expanded state during diastole phase of the heart ventricle to which the longitudinally expandable member 202 is deployed. In some instances, the longitudinally expandable member 202 can be configured to contract along the longitudinal dimension as the heart ventricle to which the longitudinally expandable member 202 is deployed contracts during systole. The longitudinally expandable member 202 can be configured to assume the compressed state during a systole phase of the heart ventricle.

The longitudinally expandable member 202 can comprise a first end 216 and a second end 218. The longitudinal dimension, such as a length, of the longitudinally expandable member 202 of the longitudinally expandable member 202 can be a linear dimension extending between opposing portions of the first and second ends 216, 218. The longitudinally expandable member 202 can assume a first length in a compressed state and a second length that is longer than the first length in an expanded state. For example, the longitudinally expandable member 202 can expand from the first length to the second length to transform from the compressed state to the expanded state. The longitudinally expandable member 202 can contract from the second length to the first length to transform from the expanded state to the compressed state. As described herein, the longitudinally expandable member 202 can be reversibly expandable along the longitudinal dimension. For example, the longitudinally expandable member 202 can contract to assume the first length during ventricular systole. During ventricular diastole, the longitudinally expandable member 202 can expand from the first length to the second length. The longitudinally expandable member 202 can then contract from the second length to the first length during a subsequent ventricular systole phase.

In some instances, the second length of the longitudinally expandable member 202 can be between about 10% to about 50% longer than the first length, including about 20% to about 50%. In some instances, the second length can be about 20% longer than the first length.

As described herein, one or more medical implant assemblies can be deployed into a heart ventricle. For example, one or more longitudinally expandable members 202 can be deployed. In some instances, one longitudinally expandable member 202 can be used. For example, a length of the longitudinally expandable member 202 can be about 25% to about 75% of a longitudinal dimension, such as a length, of the heart ventricle. In some instances, a length of the longitudinally expandable member 202 can be between about 3 centimeters (cm) and about 10 centimeters (cm). In some instances, a plurality of longitudinally expandable member 202 can be arranged along a portion of the heart ventricle wall that is about 25% to about 75% of a length of the heart ventricle. In some instances, the plurality of longitudinally expandable members 202 can be arranged along a portion of the heart ventricle wall that is about 3 centimeters (cm) to about 10 centimeters (cm). Each of the plurality of longitudinally expandable members 202 can be separate from one another, or the plurality of longitudinally expandable members 202 can form a continuous unit.

Referring again to FIGS. 3A and 3B, the longitudinally expandable member 202 can comprise a plurality of bends 220, 222, 224, 226, 228 configured to facilitate transformation between the expanded and compressed states. The bends 220, 222, 224, 226, 228 can facilitate expansion and/or compression along the longitudinal dimension between the first and second lengths. The longitudinally expandable member 202 can comprise a first portion 230, a second portion 232, a third portion 234, a fourth portion 236, a fifth portion 238 and a sixth portion 240, the adjacent ones of the portions abutting the bends 220, 222, 224, 226, 228. For example, the first and second portions 230, 232 abut the first bend 220. The second and third portions 232, 234 abut the second bend 222. The third and fourth portions 234, 236 abut the third bend 224. The fourth and fifth portions 236, 238 abut the fourth bend 226. The fifth and sixth portions 238, 240 abut the fifth bend 228. Respective acute angles formed at each of the bends 220, 222, 224, 226, 228 can decrease as the longitudinally expandable member 202 contracts to assume the compressed state. The first portion 230, second portion 232, third portion 234, fourth portion 236, fifth portion 238 and sixth portion 240 of the longitudinally expandable member 202 can be positioned closer to one another to assume the compressed state, for example the first portion 230, second portion 232, third portion 234, fourth portion 236, fifth portion 238 and sixth portion 240 assuming a folded configuration. Respective acute angles formed at each of the bends 220, 222, 224, 226, 228 can increase as the longitudinally expandable member 202 expands to assume the expanded state. The first portion 230, second portion 232, third portion 234, fourth portion 236, fifth portion 238 and sixth portion 240 of the longitudinally expandable member 202 can be positioned further away from one another to assume the expanded state, for example the first portion 230, second portion 232, third portion 234, fourth portion 236, fifth portion 238 and sixth portion 240 assuming an unfolded configuration. In some instances, the longitudinally expandable member 202 can comprise an accordion configuration. In some instances, the longitudinally expandable member 202 can comprise a wave configuration. For example, the first bend 220, third bend 224, and fifth bend 228 can form crests of the wave. The second bend 222 and the fourth bend 226 can form the troughs of the wave.

The medical device assembly 200 can comprise a plurality of anchors configured to secure the longitudinally expandable member 202 to the heart ventricle. In some instances, the medical device assembly 200 can comprise a first anchor and a second anchor. The anchors are not shown in FIGS. 3A and 3B for simplicity. The first anchor can be configured to be coupled to a first portion of the longitudinally expandable member 202 to secure the first portion of the longitudinally expandable member 202 to a first location on the ventricular heart wall surface. The second anchor can be configured to be coupled to a second portion of the longitudinally expandable member 202 to secure the second portion of the longitudinally expandable member 202 to a second location on the ventricular heart wall surface. In some instances, the longitudinally expandable member 202 can comprise a first anchor engagement feature 212 on a first distal portion 208 and a second anchor engagement feature 214 on a second distal portion 210. In some instances, the first and second engagement features 212, 214 can each comprise an opening configured to receive a respective anchor. For example, the first and second distal portions 208, 210 can each define an opening extending through the longitudinally expandable member 202. A portion of a respective anchor can be received within an opening to facilitate securing the longitudinally expandable member 202 to the wall of the heart ventricle.

The longitudinally expandable member 202 can be secured to the heart ventricle wall surface such that a first surface 204 of the longitudinally expandable member 202 can be oriented toward the ventricular heart wall surface and a second surface 206 can be oriented away from the ventricular heart wall surface. In some instances, the longitudinally expandable member 202 can comprise a wave spring, including a linear wave spring. A first surface of the linear wave spring can be configured to be oriented toward the ventricular heart wall surface. A second surface of the linear wave spring can be configured to be oriented away from the ventricular heart wall surface.

In some instances, a longitudinally expandable member can comprise a shape memory material. In some instances, a longitudinally expandable member can comprise nitinol. In some instances, a longitudinally expandable member can be a nitinol longitudinally expandable member. Alternatively or in combination, a longitudinally expandable member can comprise an electrically activated polymer. For example, application of an electrical signal to the electrically activated polymer can facilitate transformation of the longitudinally expandable member between an expanded state and a compressed state. In some instances, a longitudinally expandable member can be an electrically activated polymer longitudinally expandable member.

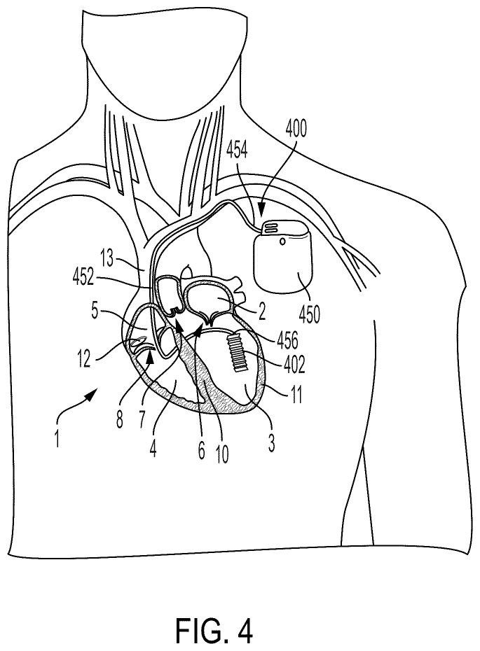

FIG. 4 shows a cut-away view of a heart 1 and an example of a medical device assembly 400 that includes a longitudinally expandable member 402 comprising an electrically activated polymer, where the longitudinally expandable member 402 is deployed to the left ventricle 3. The longitudinally expandable member 402 is shown secured to a portion of a ventricular wall 11 of the left ventricle 3. Any number of electrically activated polymers can be used, including one or more electroactive polymers. The medical device assembly 400 can comprise a housing 450 configured to receive one or more other components of the assembly 400 to facilitate implantation of the components. For example, the housing 450 can be configured to receive a generator (not shown) for generating an electrical signal to activate the electrically activated polymer. A controller (not shown) can be received in the housing 450 and configured to control generation of electrical signals for activating the electrically activated polymer. The controller can be configured to receive and/or process various signals for determining triggering of the generator. In some instances, the medical device assembly 400 can comprise a cardiac cycle monitor (not shown) configured to monitor the cardiac cycle of the heart. The cardiac cycle monitor can be received within the housing 450. The cardiac cycle monitor can be in communication with the controller such that the controller can send a trigger signal to the generator based on the cardiac cycle of the heart, such as in response to start of ventricular diastole and/or ventricular systole. The generator can generate an electrical signal for activating the electrically activated polymer in response to the trigger signal. In some instances, the generator can be configured to generate an electrical signal for triggering expansion and/or lengthening of the electrically activated polymer in response to the heart ventricle entering a diastole phase. In some instances, the generator can be configured to generate an electrical signal for triggering contraction and/or shortening of the electrically activated polymer in response to the heart ventricle entering a systole phase. The longitudinally expandable member 402 can be configured to transform between the expanded and contracted states on a cadence synchronized with the heart rate, including with every heartbeat or during another specified period. For example, transformation of the longitudinally expandable member 402 can be triggered during periods of elevated left atrial pressure. In some instances, various components of the medical device assembly 400 can be battery powered. Alternatively or in combination, various components of the medical device assembly 400 can be extra-corporeally powered and/or charged.

The housing 450 can be implanted at a target location under the skin. A wire 452 can extend between the housing 450, such as the generator received within the housing 450, and the longitudinally expandable member 402 for transmitting the electrical signal from the generator to the longitudinally expandable member 402. For example, a first end portion 454 of the wire 452 can be coupled to the generator received within the housing 450. The wire 452 can comprise at least a portion configured to be disposed within the heart ventricle to be coupled to the longitudinally expandable member 402. For example, a second end portion 456 of the wire 452 can be coupled to the longitudinally expandable member 402 disposed within the heart ventricle. In some instances, an electrode can be coupled to the wire 452. The electrode can be configured to be disposed within the heart ventricle and in contact with the longitudinally expandable member 402 to deliver the electrical signal for electrically activating the longitudinally expandable member 402.

As described herein, the housing 450 can be implanted into a pocket under the skin. For example, a subcutaneous pocket can be formed, including in the left infraclavicular region. A portion of the wire 452 can be advanced into a vein, including a subclavian vein, cephalic vein, or jugular vein. In some instances, the wire 452 can then be advanced into the superior vena cava 13, and from the superior vena cava 13 into the right atrium 5. In some instances, the wire 452 can be advanced through the tricuspid valve 8 and into the right ventricle 4. The wire 452 can then be disposed through the septal wall 10 from the right ventricle 4 into the left ventricle 3 such that the second end portion 456 can be coupled to the longitudinally expandable member 402. Alternatively, access to the longitudinally expandable member 402 can be provided using an atrial transseptal approach.

The longitudinally expandable member 402 can have one or more other characteristics of the longitudinally expandable member 202 described with reference to FIGS. 2 and 3. For example, remaining features of the longitudinally expandable member 402 can be similar to or the same as those of the longitudinally expandable member 202. In some instances, the longitudinally expandable member 402 comprising the electrically activated polymer can be used in patients with advanced heart failure.

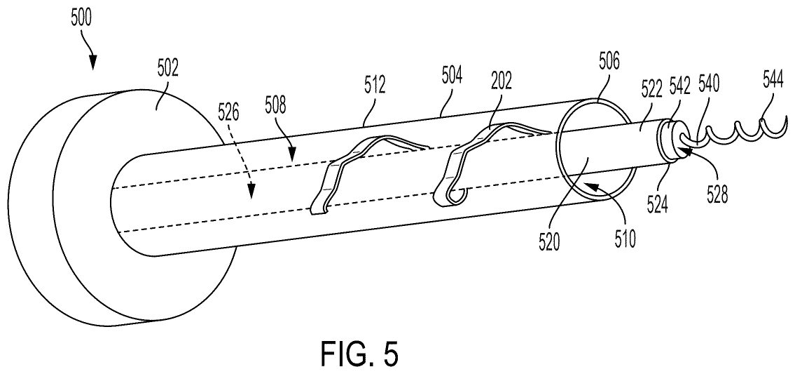

FIG. 5 is a perspective view of an example of a delivery assembly 500 configured to deliver one or more medical device assemblies described herein, including for example, medical device assemblies 200, 400 described with reference to FIGS. 2 through 4. FIG. 5 shows use of the delivery assembly 500 to deliver the longitudinally expandable member 202 described with reference to FIGS. 2 and 3. The delivery assembly 500 can comprise a proximal handle 502 and an elongate shaft 504 extending distally from the proximal handle 502. The elongate shaft 504 can comprise a first delivery lumen 508 extending along a longitudinal axis of the elongate shaft 504. The first delivery lumen 508 can be configured to receive the longitudinally expandable member 202. The elongate shaft 504 can comprise a second delivery lumen 526 extending along the longitudinal axis. The second delivery lumen 526 can be configured to receive one or more anchors configured to couple the longitudinally expandable member 202 to a ventricular heart wall. In some instances, the first delivery lumen 508 can be disposed around the second delivery lumen 526. Alternatively, the first delivery lumen 508 can be adjacent to the second delivery lumen 526. For example, both delivery lumens 508, 526 can extend along the longitudinal axis of the elongate shaft 504 and the first delivery lumen 508 being to a side of the second delivery lumen 526.

FIG. 5 shows the first delivery lumen 508 being disposed around the second delivery lumen 526. The elongate shaft 504 can comprise an outer shaft portion 512 at least partially defining the first delivery lumen 508. An inner shaft portion 520 of the elongate shaft 504 can define at least in part the second delivery lumen 526. The inner shaft portion 520 can extend within the outer shaft portion 512. In some instances, the inner shaft portion 520 can be coaxial with the outer shaft portion 512, for example both extending along the longitudinal axis of the elongate shaft 504. The first delivery lumen 508 can be configured to receive the longitudinally expandable member 202 arranged in a helical configuration therewithin. For example, the longitudinally expandable member 202 can be arranged around a portion of the inner shaft portion 520 to assume the helical configuration. In some instances, a plurality of longitudinally expandable members 202 can be received within the first delivery lumen 508, each of the longitudinally expandable members 202 being at a respective position along the longitudinal dimension of the first delivery lumen 508. The plurality of longitudinally expandable members 202 can be preloaded within the first delivery lumen 508.

As described herein, the longitudinally expandable member 202 can be coupled to a ventricular heart wall using a plurality of anchors. An example of an anchor 540 is shown in FIG. 5. A portion of the anchor 540 is shown as extending out of a distal end 524 of the inner shaft portion 520 through the distal opening 528. In some instances, one or more anchors 540 can be preloaded within the second delivery lumen 526. For example, the plurality of anchors can be arranged one after the other in the second delivery lumen 526. Alternatively, each anchor 540 can be advanced through the second delivery lumen 526 after a portion of the longitudinally expandable member 202 is at a target position. For example, one anchor 540 can be preloaded within the second delivery lumen 526.

The anchor 540 can comprise a spiral portion 544. In some instances, the anchor 540 can comprise a corkscrew configuration. A proximal portion 542 of the anchor 540 can be configured to be engaged with the longitudinally expandable member 202. At least a portion of the spiral portion 544 can be configured to be embedded into the ventricular heart wall. For example, the anchor 540 can comprise a helical portion configured to be screwed into the ventricular heart wall. The anchor 540 can engage with the first anchor engagement feature 212 of the longitudinally expandable member 202. In some instances, the spiral portion 544 can be advanced through an opening on the first distal portion 208 of the longitudinally expandable member 202 configured to receive the anchor 540 such that a portion of the longitudinally expandable member 202 can be positioned between the proximal portion 542 of the anchor 540 and a portion of the ventricular heart wall. A second anchor 540 can be deployed to engage with the second anchor engagement feature 212 on the second distal portion 210 of the longitudinally expandable member 202. For example, a spiral portion 544 of the second anchor 540 can be advanced through an opening on the second distal portion 210 such that another portion of the longitudinally expandable member 202 can be positioned between the proximal portion 542 of the second anchor 540 and another portion of the ventricular heart wall.

A process for deploying the longitudinally expandable member 202 can comprise advancing a portion of the longitudinally expandable member 202 out of the first delivery lumen 508 through the distal opening 510 at the distal end 506 of the outer shaft portion 512. A first portion of the longitudinally expandable member 202 can be positioned at a first position on the ventricular heart wall surface. For example, a first distal portion 208 (not shown) can be positioned at a first position on the ventricular heart wall surface. In some instances, the inner shaft portion 520 can be translated distally relative to the outer shaft portion 512, or the outer shaft 512 can be translated proximally relative to the inner shaft portion 520, to facilitate deployment of the longitudinally expandable member 202 and anchors 540 for securing the longitudinally expandable member 202. FIG. 5 shows a distal portion 522 of the inner shaft portion 520 disposed distally of the distal opening 510. In some instances, the inner shaft portion 520 can be translated distally relative to the outer shaft portion 512 after the first portion of the longitudinally expandable member 202 is positioned. The first portion of the longitudinally expandable member 202 can be positioned at a first location on the ventricular heart wall surface. The anchor 540 can be positioned out of the distal opening 528 of the inner shaft portion 520 to be advanced through the first portion, such as through the first anchor engagement feature 212 of the first end portion 208, of the longitudinally expandable member 202 and into the first location of the heart ventricular wall surface. At least a portion of the first portion of the longitudinally expandable member 202 can sandwiched between the proximal portion 542 of the anchor 540 and the ventricular heart wall to secured to the first portion of the longitudinally expandable member 202 to the ventricular heart wall. The longitudinally expandable member 202 can be deployed during ventricular systole, such as while the longitudinally expandable member 202 is in a compressed state such that the longitudinally expandable member 202 can be biased to stretch and/or lengthen the portion of the ventricular heart wall.

In some instances, a second anchor can be used to secure the longitudinally expandable member 202 to the ventricular heart wall. A second portion of the longitudinally expandable member 202 can be advanced out of the first delivery lumen 508 and positioned at a target location on the ventricular heart wall surface. For example, a second distal portion 210 comprising a second anchor engagement feature 214 can be advanced out of the first delivery lumen 508 through the distal opening 510 and positioned on a second location of the heart ventricular wall surface. The second anchor 540 can be advanced out of the second delivery lumen 526 and through the second portion of the longitudinally expandable member 202, such as the second anchor engagement feature 214 and into a second location of the heart ventricular wall surface.

In some instances, the delivery assembly 500 can comprise one or more components configured to deploy an anchor to the ventricular heart wall. For example, one or more components can be configured to screw one or more anchors into the ventricular heart wall. For example, the delivery assembly 500 can be configured to screw the anchor 540 through the first portion of the longitudinally expandable member 202 and/or into the ventricular heart wall at the first location. The delivery assembly 500 can be configured to screw the second anchor through the second portion of the longitudinally expandable member 202 and/or into the ventricular heart wall at the second location. In some instances, the delivery assembly 500 can comprise a rod (not shown) configured to engage with an anchor 540 and rotate the anchor 540 to screw the anchor 540 into a target location. For example, the rod can comprise a distal end portion configured to engage with the anchor 540, including a proximal portion 542 of the anchor 540. The rod can comprise an elongate shaft and a distal end portion configured to engage with the anchor 540. The rod can be configured to be advanced the anchor 540 through the second delivery lumen 526. The rod can be configured to rotate the anchor 540 around a longitudinal axis of the anchor 540 to screw at least a portion of the spiral portion 544 of the anchor 540 through the first portion of the longitudinally expandable member 202 and into the ventricular heart wall.

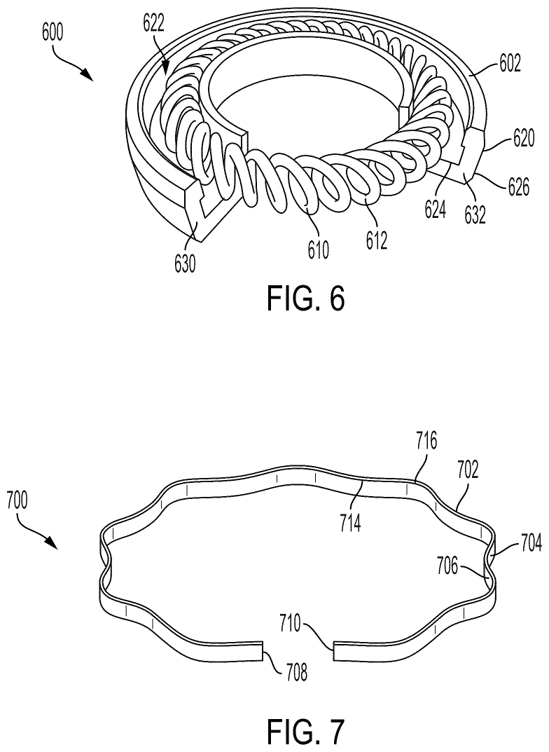

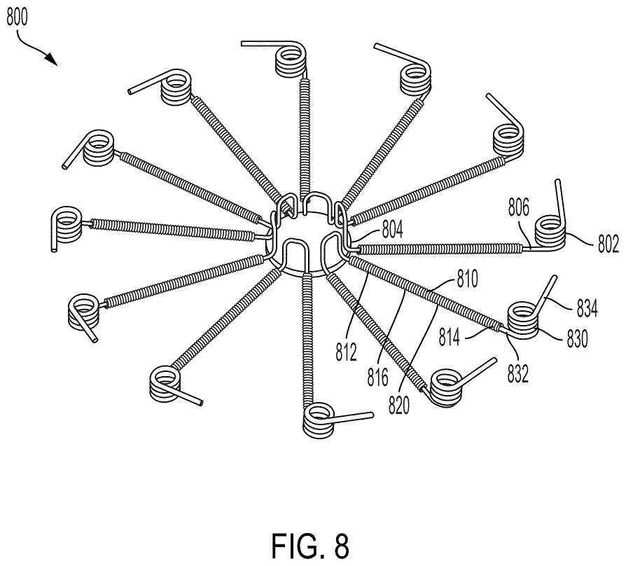

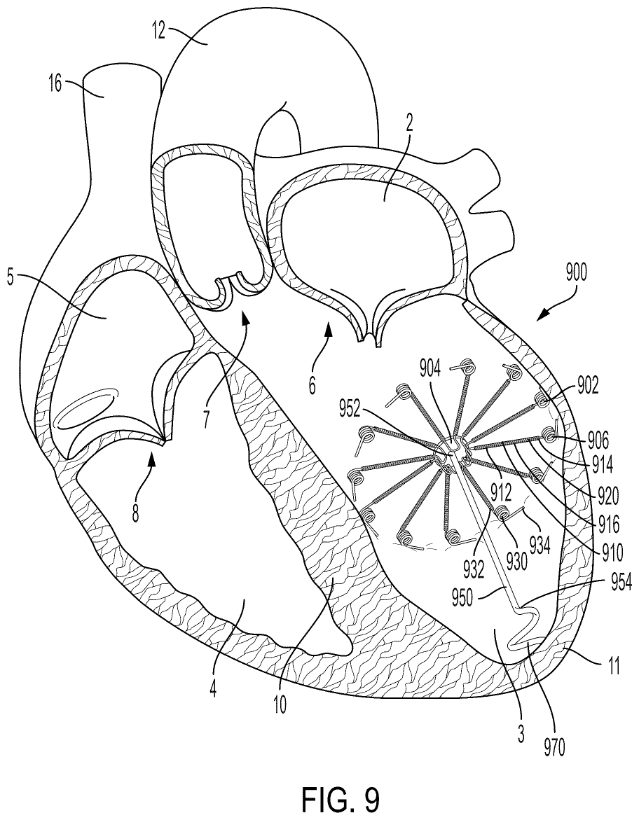

In some instances, circumferential expansion and/or lengthening of myocardial muscle fibers can be augmented. FIGS. 6, 7, 8 and 9 describe various examples of medical implant devices 600, 700, 800, 900 comprising a circumferentially disposed portion configured to be reversibly expandable radially. The circumferentially disposed portion can be configured to be received within a heart ventricle. Respective portions of the circumferentially disposed portion can be secured to the ventricular heart wall. In some instances, the circumferentially disposed portion can assume a radially expanded state during at least a portion of a diastole phase of the heart ventricle. In some instances, the circumferentially disposed portion can assume a radially collapsed state during at least a portion of a systole phase of the heart ventricle. A plurality of anchors can be used secure the circumferentially disposed portion to the ventricular heart wall. The anchors are not shown in FIGS. 8 through 9 for simplicity. The medical implant devices 600, 700, 800, 900 described with reference FIGS. 6 through 10 can be positioned over a portion of a ventricular heart wall surface aligned with an axis of a subendocardial circumferential fiber of the ventricular heart wall. In some instances, a circumferentially disposed portion of one or more of the medical implant devices 600, 700, 800, 900 can be configured to be secured to a circumferential portion of the ventricular heart wall and be aligned with subendocardial circumferential fibers of the ventricular heart wall. For example, the orientation of the circumferentially disposed portions can be the same as or similar to that of the subendocardial circumferential fibers. In some instances, the circumferentially disposed portion of one or more of the medical implant devices 600, 700, 800, 900 can be configured to augment radial expansion during a diastole phase of the heart ventricle, including during an early and/or late phase of diastole. In some instances, a circumferentially disposed portion of one or more of the medical implant devices 600, 700, 800, 900 can be secured to a circumferential portion at an Infra-annular location on the ventricular heart wall. In some instances, a circumferentially disposed portion of one or more of the medical implant devices 600, 700, 800, 900 can be configured to be secured to a circumferential portion of the ventricular heart wall at an apical location, including an apical region of the heart. In some instances, a circumferentially disposed portion of one or more of the medical implant devices 600, 700, 800, 900 can be configured to be secured to a circumferential portion of the ventricular heart wall inferior of and adjacent to papillary muscles of the heart ventricle.

As used herein, the “apical region” can include the inferior tip of the heart. The inferior tip is referred to herein as the apex of the heart and is generally located on the midclavicular line, in the fifth intercostal space. The apex can be considered part of the greater apical region. Generally, the apical region of the heart can be a bottom region of the heart that is within the left or right ventricular region but is distal to the mitral and tricuspid valves and toward the tip of the heart. More specifically, the apical region may be considered to comprise a bottom portion of the heart that is within about 20 centimeters (cm) to the right or to the left of the median axis of the heart.

FIG. 6 is a perspective view of an example of a medical implant device 600 comprising a circumferentially disposed portion 602 configured to be reversibly expandable radially. The circumferentially disposed portion 602 can be secured to a circumferential portion of a ventricular heart wall of a heart ventricle, such as a ventricular heart wall surface oriented toward the heart ventricle. The circumferentially disposed portion 602 can comprise a coil spring 610, such as a linear coil spring, comprising a plurality of coils 612. In some instances, the coil spring 610 can be a ring-shaped coil spring. The coil spring 610 can be reversibly expandable radially. For example, the coil spring 610 can assume a radially expanded state during at least a portion of a diastole phase of the heart ventricle, and a radially collapsed state during at least a portion of a systole phase of the heart ventricle. The circumferentially disposed portion 602 can be configured to be biased toward the radially expanded state to supplement radial expansion and/or untwisting of the heart ventricle. In the radially expanded state, coils 612 of the coil spring 610 can be in a relaxed state, for example such that the ring formed by the coil spring 610 has a diameter larger than that in the radially collapsed state. In the radially collapsed state, the coils 612 of the coil spring 610 can be in a tensioned and/or compressed state.

The circumferentially disposed portion 602 can comprise a partial ring-shaped member 620 configured to receive the coil spring 610. For example, at least a portion of the coil spring 610 can be received by the partial ring-shaped member 620 to facilitate securing the coil spring 610 to the ventricular heart wall. A plurality of anchors (not shown) can be used to secure the partial ring-shaped member 620 to the ventricular heart wall, including a ventricular heart wall surface oriented toward the heart ventricle. In some instances, the partial ring-shaped member 620 can comprise a recess 622 configured to receive at least a portion of the coil spring 610. Alternatively or in combination, the coil spring 610 can be at least partially encased in a partial ring-shaped member. For example, the partial ring-shaped member can comprise a tubular configuration defining a lumen configured to receive the coil spring 610. The partial ring-shaped member 620 can comprise a biocompatible material, including a synthetic and/or biological material which promotes integration into and healing with the ventricular heart wall. In some instances, the partial ring-shaped member 620 can comprise a polyester material, including a thermoplastic polyester resin, such as polyethylene terephthalate (PET) (e.g., Dacron™).

A first surface 624 of the partial ring-shaped member can define the recess 622. In some instances, the first surface 624 can define a recess comprising a superior orientation. For example, the recess 622 can have a superior orientation while the medical implant device 600 is deployed into the heart ventricle. A second surface 626 of the partial ring-shaped member can comprise a portion configured to be oriented toward the heart ventricular wall. For example, the portion of the second surface 626 oriented toward the heart ventricular wall can be over and in contact with the heart ventricle wall surface when the medical implant device 600 is secured to the heart ventricular wall. In some instances, a cross-section of the partial ring-shaped member can have a “U” shape.

The partial ring-shaped member 620 can comprise a partial-ring configuration. The partial ring-shaped member 620 can be discontinuous. The partial ring-shaped member 620 can comprise a first end 630 and a second end 632. The separation distance between the first end 630 and the second end 632 of the partial ring-shaped member 620 can facilitate its radial expansion and radial compression. For example, the separation distance between the first end 630 and the second end 632 can increase as the heart ventricle relaxes during diastole and decrease as the heart ventricle contracts during systole. The separation distance between the first end 630 and the second end 632 can change to accommodate the coil spring 610 as the coil spring transforms between the radially expanded and radially collapsed states. In some instances, FIG. 6 can show the coil spring 610 and the partial ring-shaped member 620 in relaxed states, for example while radially expanded, such that the coil spring 610 and the partial ring-shaped member 620 can be compressed during ventricular systole to assume tensioned states. The coil spring 610 and the partial ring-shaped member 620 can transform back to the relaxed states during ventricular diastole.

FIG. 7 is a perspective view of another example of a medical implant device 700 comprising a circumferentially disposed portion 702 configured to be reversibly expandable radially. The circumferentially disposed portion 702 can be configured to be secured to a heart ventricle. For example, the circumferentially disposed portion 702 can be configured to be secured to a circumferential portion of a heart ventricle wall surface oriented toward the heart ventricle. The circumferentially disposed portion 702 can assume a radially expanded state during at least a portion of a diastole phase of the heart ventricle, and a radially collapsed state during at least a portion of a systole phase of the heart ventricle. The circumferentially disposed portion 702 can be configured to be biased toward the radially expanded state to supplement radial expansion and/or untwisting of the heart ventricle. For example, the radially expanded state is a relaxed state for the circumferentially disposed portion 702. A first surface 704 of the circumferentially disposed portion 702 can be configured to be oriented toward the ventricular heart wall surface. A second surface 706 of the circumferentially disposed portion 702 can be configured to be oriented away from the ventricular heart wall surface. In some instances, the circumferentially disposed portion 702 can be secured to the ventricular heart wall such that the first surface 704 is over and in contact with the ventricular heart wall.

The circumferentially disposed portion 702 can be discontinuous. For example, the circumferentially disposed portion 702 can comprise a partial ring configuration. In some instances, the circumferentially disposed portion 702 can comprise a first end 708 and a second end 710. The separation distance between the first end 708 and the second end 710 of the circumferentially disposed portion 702 can increase as the heart ventricle relaxes during diastole and decrease as the heart ventricle contracts during systole. The circumferentially disposed portion 702 can assume a partial ring configuration such that the discontinuity of the circumferentially disposed portion 702 can facilitate radial expansion and compression of the circumferentially disposed portion 702. For example, the circumferentially disposed portion 702 is shown in FIG. 7 as assuming the radially expanded state, or while relaxed, such as for ventricular diastole. The separation distance between the first end 708 and the second end 710 can decrease as the circumferentially disposed portion 702 assumes the radially collapsed state, such as while the heart contracts during ventricular systole.

In some instances, the circumferentially disposed portion 702 can comprise a plurality of bends. The plurality of bends can have alternating orientations. For example, the bends can alternate between convex and concave orientations. In some instances, the circumferentially disposed portion 702 can comprise a wave configuration. For example, a first bend 714 can be a crest of the wave and a second bend 716 adjacent to the first bend 714 can be a trough of the wave. Reversible deformation of the plurality of bends can provide at least in part the reversible radial expansion and contraction of the circumferentially disposed portion 702. The plurality of bends can transform between an undeformed and/or relaxed state and a deformed, compressed and/or tensioned state as the heart ventricle transitions between diastole and systole, respectively.

In some instances, the circumferentially disposed portion 702 can comprise a linear wave spring. For example, the linear wave spring can form a partial ring. A first surface of the linear wave spring can be configured to be oriented toward the ventricular heart wall surface. A second surface of the linear wave spring can be configured to be oriented away from the ventricular heart wall surface. The first surface can be over and in contact with the ventricular heart surface.

FIG. 8 is a perspective view of an example of a medical implant device 800 that includes a circumferentially disposed portion 802 comprising a plurality of radially extending spokes 810. In some instances, the medical implant device 800 can be configured to supplement radial expansion and/or untwisting of a target heart ventricle. A first end portion 812 of each of the plurality of radially extending spokes 810 can be configured to be oriented toward a center of the circumferentially disposed portion 802. For example, each of the first end portions 812 can be oriented toward a central portion 804 of the circumferentially disposed portion 802. A second end portion 814 of each of the plurality of radially extending spokes 810 can be configured to be oriented toward an edge portion 806 of the circumferentially disposed portion 802. In some instances, one or more of the radially extending spokes 810 can comprise a coil spring 820, such as a linear coil spring, along at least a portion of a length of the radially extending spoke 810. The coil spring 820 can extend along a longitudinal axis of the radially extending spoke 810, for example such that the longitudinal axis of the radially extending spoke 810 is coaxial and/or parallel or substantially parallel to that of a longitudinal axis of the coil spring 820. The longitudinal axis of the radially extending spoke 810 can extend between the first and second end portions 812, 814 of the spoke 810. In some instances, a radially extending spoke 810 can comprise one or more coil springs along a portion thereof, including a first end portion 812, mid-portion 816 and/or second end portion 814 of the radially extending spoke 810. In some instances, a radially extending spoke 810 can comprise a coil spring 820 along the entire or substantially entire length thereof. FIG. 8 shows each of the plurality radially extending spokes 810 comprising a coil spring 820, such as a linear coil spring, extending along at least a portion of a length of the radially extending spoke 810. In some instances, each of the plurality radially extending spokes 810 can comprise a coil spring 820, such as a linear coil spring, extending along an entire or substantially entire length thereof. The plurality of coil springs 820 can provide increased radial expansion of the heart ventricle, for example augmenting radial expansion of the circumferential subendocardial fibers. One or more of the coil springs 820 can be in a relaxed state, such as an expanded state, during ventricular diastole, and a compressed state during ventricular systole. For example, the one or more coil springs 820 can transform between the relaxed and compressed states as the heart ventricle transitions between diastole and systole, respectively. In some instances, not all of the spokes 810 have a coil spring 820. For example, spokes 810 comprising second end portions 814 configured to be positioned at or proximate to corners of the interventricular septum and lateral free wall of the heart ventricle, such as the left heart ventricle, can comprise the coil spring 820.

In some instances, the circumferentially disposed portion 802 can comprise a plurality of torsion springs 830 around the edge portion 806. Referring again to FIG. 8, a respective torsion spring 830 can be coupled to the second end portion 814 of each of the radially extending spokes 810. Each of the torsion springs 830 can be disposed at respective positions around the edge portion 806. Each of the plurality of torsion springs 830 can have a first end portion 832 configured to be coupled to the second end portion 814 of a respective one of the plurality of radially extending spokes 810. A second end portion 834 of each of the torsion springs 830 can be configured to be coupled to a respective portion of the ventricular heart wall. The torsion springs 830 can be configured to facilitate untwisting of the ventricle. For example, each of the torsion springs 830 can be oriented such that the torsion springs 830 are biased toward an expanded heart ventricle, the torsion springs 830 being in a relaxed state during a ventricular diastole phase and a compressed state during a ventricular systole phase. In some instances, a longitudinal axis of each of the torsion springs 830 can be perpendicular or substantially perpendicular to the plane within which the plurality of radially extending spokes 810 extend. The longitudinal axis of each of the torsion springs 830 can be perpendicular or substantially perpendicular relative to a plane within which each respective radially extending spoke 810 extends, including the longitudinal axis of the respective radially extending spoke 810. The torsion springs 830 can transform between the relaxed state and compressed state as the heart ventricle transitions between the diastole phase and the systole phase, respectively. In some instances, FIG. 8 can show the coil springs 820 and the torsion springs 830 in compressed states, such that the coil springs 820 and the torsion springs 830 can relax during ventricular diastole and assume relaxed states. The coil springs 820 and torsion springs 830 be compressed and transform back to the compressed states during ventricular systole.

In some instances, the circumferentially disposed portion 802 can optionally comprise a central torsion spring (not shown). The central torsion spring can be disposed in the central portion 804 of the circumferentially disposed portion 802. The central torsion spring can be configured to facilitate untwisting of the heart ventricle. For example, the central torsion spring can be oriented such that the central torsion spring can be biased toward an expanded heart ventricle. The central torsion spring can be in a relaxed state during a ventricular diastole phase and a compressed state during a ventricular systole phase. For example, the central torsion spring can transform between the relaxed state and compressed state as the heart ventricle transitions between the diastole phase and the systole phase, respectively. In some instances, a longitudinal axis of the central torsion spring can be perpendicular or substantially perpendicular to the plane within which the plurality of radially extending spokes 810 extend. The central torsion spring can comprise a respective end portion coupled to a plurality of the radially extending spokes 810. In some instances, the central torsion spring can comprise end portions coupled to each of the plurality of radially extending spokes 810, directly or indirectly. In some instances, the central torsion spring can comprise a respective end portion coupled to each of the plurality of radially extending spokes 810.

FIG. 9 shows an example of a medical implant device 900 comprising circumferentially disposed portion 902 and a rod 950 coupled to the circumferentially disposed portion 902. In some instances, the medical implant device 900 can be configured to supplement radial expansion and/or untwisting of a target heart ventricle. A first end portion 952 of the rod 950 can be coupled to the circumferentially disposed portion 902. In some instances, the first end portion 952 can be coupled to a central portion 904 of the circumferentially disposed portion 902. A second end portion 954 of the rod 950 can be coupled to an anchor 970. In some instances, the anchor 970 can have a spiral and/or helical portion. For example, the anchor 970 can have a corkscrew configuration. For example, the anchor 970 can comprise a stainless steel and/or nitinol tissue screw. As described in further detail herein, one or more portions of the rod 950 can bend to allow the rod 950 to assume a configuration having a shorter longitudinal dimension. In some instances, while the rod 950 is in an extended configuration, the rod 950 and/or the anchor 970 can comprise a perpendicular or substantially perpendicular orientation relative to the circumferentially disposed portion 902. In some instances, a longitudinal axis, such as an axis extending between the first and second end portions 952, 954, of the rod 950 can be perpendicular or substantially perpendicular to a plane in which the circumferentially disposed portion 902 extends. For example, while the rod 950 is unbent, both the rod 950 and the anchor 970 can comprise a perpendicular or substantially perpendicular orientation relative to the circumferentially disposed portion 902. In some instances, the rod 950 can comprise a deflectable rod, cable and/or wire. Alternatively or in combination, the rod 950 can comprise a coil spring, including a linear coil spring. The rod 950 can comprise any number of different materials, including a polymer and/or a shape-memory alloy, such as nitinol.

FIG. 9 shows the medical implant device 900 deployed into a heart 1. The medical implant device 900 can be positioned within a heart ventricle. For example, the medical implant device 900 can be deployed into the left ventricle 3. Respective portions of the circumferentially disposed portion 902 can be secured to the ventricular heart wall 11. The circumferentially disposed portion 902 can be coupled to portions of the ventricular heart wall 11 as described herein. The anchor 970 can be coupled to another portion of the ventricular heart wall 11, such as a portion of the ventricular heart wall 11 in an apical region of the heart 1. At least a portion of the anchor 970 can be embedded within the ventricular heart wall 11. For example, at least a portion of the anchor 970 can be screwed into a portion of the ventricular heart wall 11 in the apical region of the heart. FIG. 9 shows the anchor 970 perpendicular or substantially perpendicular orientation relative to the circumferentially disposed portion 902.

The circumferentially disposed portion 902 can comprise one or more features of the circumferential portion 802 described with reference to FIG. 8. For example, the circumferentially disposed portion 902 can comprise a plurality of radially extending spokes 910. A first end portion 912 of each of the plurality of radially extending spokes 910 can be configured to be oriented toward a central portion 904 of the circumferentially disposed portion 902. A second end portion 914 of each of the plurality of radially extending spokes 910 can be configured to be oriented toward an edge portion 906 of the circumferentially disposed portion 902. In some instances, one or more of the radially extending spokes 910 can comprise a coil spring 920, such as a linear coil spring, along at least a portion of a length of the radially extending spoke 910. In some instances, a radially extending spoke 910 can comprise one or more coil springs along a portion thereof, including the first end portion 912, mid-portion 916 and/or second end portion 914 of the radially extending spoke 910. Each of a plurality of torsion springs 930 can be disposed at respective positions around the edge portion 906, comprising a first end portion 932 configured to be coupled to the second end portion 914 of a respective one of the plurality of radially extending spokes 910. A second end portion 934 of each of the torsion springs 930 can be configured to be coupled to a respective portion of the ventricular heart wall 11. In some instances, the circumferentially disposed portion 902 can optionally comprise a central torsion spring.

Twisting of the heart apex can contribute to ventricular ejection, such as left ventricular ejection. Untwisting of the heart apex can contribute to relaxation of the left ventricle. Changes in torsion can have significant effects on atrial pressure. Anchoring the circumferentially disposed portion 902 to the apical region of the heart can be configured to provide a baseline level of torsion. The baseline level of torsion can be biased toward untwisting of the heart ventricle. In some instances, anchoring of the anchor 970 can be adjusted to provide a desired baseline level of torsion. As described in further detail herein, the rod 950 can be configured to deflect and/or bend to accommodate the torsion in systolic compression. The rod 950 can be configured to unbend and/or straighten during diastole such that the torsion is re-exerted. Bending and/or deflection of the rod 950 can unload the spring, reducing impact of the medical implant device 900 on systolic force and wall stress of the heart as the apex moves towards the base during contraction. Unbending and/or straightening of the rod 950 during diastole can facilitate exertion of rotational force in the desired direction by the rod 950, thus promoting diastolic untwisting of the heart ventricle. Improved diastolic untwisting can provide greater left atrial emptying via improved conduit function.

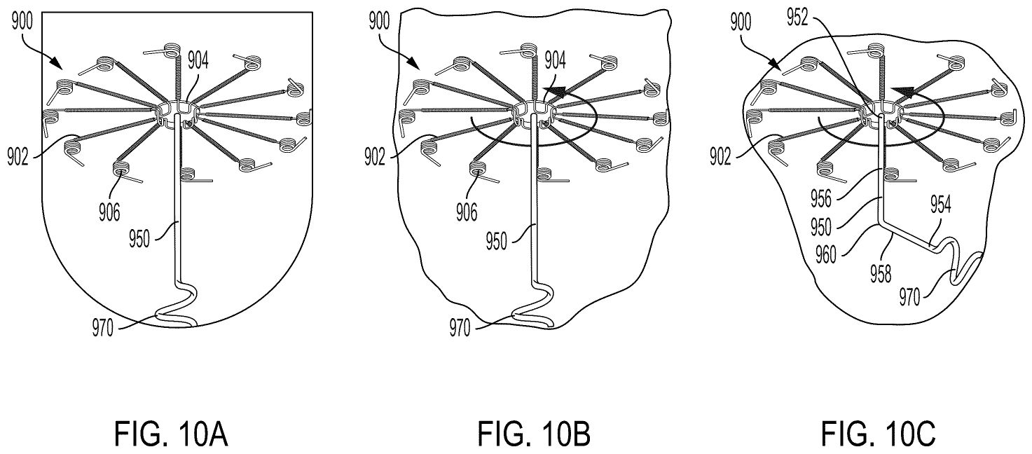

FIGS. 10A, 10B and 10C each show the medical implant device 900 described with reference to FIG. 9 deployed to a heart ventricle and in various states while the heart ventricle transitions from a diastole phase to a systole phase. FIG. 10A shows the medical implant device 900 in a relaxed state while the heart ventricle in the diastole phase. FIG. 10B shows the medical implant device 900 in a first compressed state as the heart ventricle begins to contract during the systole phase. FIG. 10C shows the medical implant device 900 in a second compressed state as the assumes a fully compressed state during the systole phase. Referring to FIG. 10A, at least a portion of the rod 950 can be configured to be oriented perpendicularly or substantially perpendicularly relative to a plane of the circumferentially disposed portion 902. For example, the entire or substantially entire length of the rod 950 can be configured to be oriented perpendicularly or substantially perpendicularly during at least a portion of the diastole phase of the heart ventricle, such as during the entire or substantially entire diastole phase. In some instances, the plurality of coil springs 920 can be in a relaxed state. The plurality of coil springs 920 can be uncompressed.

Referring to FIG. 10B, as the heart ventricle begins to contract, the medical implant device 900 can assume the first compressed state. For example, during at least a portion of early systole, at least a portion of the circumferentially disposed portion 902 can be rotated around an axis oriented perpendicularly or substantially perpendicularly relative to a plane of the circumferentially disposed portion 902, such as relative to its position during the diastole phase. In some instances, during at least a portion of early systole, at least a portion of the circumferentially disposed portion 902 can be rotated relative to the rod 950. In some instances, during at least a portion of early systole, at least a portion of the circumferentially disposed portion 902 and the rod 950 can be rotated, such as relative to their positions during the diastole phase. In some instances, at least a portion of the rotation can be provided by the plurality of torsion springs 930. For example, the plurality of torsion springs 930 can assume a tensioned state while the circumferentially disposed portion 902 is in the rotated position. In some instances, at least a portion of the rotation can be provided by a central torsion spring. Rotation of the circumferentially disposed portion 902 can be configured to accommodate the contraction of the ventricle during systole. The rod 950 is shown as being in an unbent configuration. For example, the rod 950 can be oriented perpendicularly or substantially perpendicularly relative to the plane of the circumferentially disposed portion 902. In some instances, the plurality of coil springs 920 can be compressed, for example in a first compressed state. Alternatively, the plurality of coil springs 920 can be in a relaxed state.

Referring to FIG. 10C, the heart ventricle is shown as being in a fully compressed state, such as during late systole. The rod 950 can assume a bent and/or deflected configuration. At least a portion of the rod 950 can be configured to be oriented obliquely relative to the plane of the circumferentially disposed portion 902 during at least a portion of the systole phase of the heart ventricle. For example, one or more portions of the rod 950 can be configured to assume a bend. FIG. 10C shows the rod 950 assuming a bend 956. The bend 956 can allow the medical implant device 900 to accommodate further contraction of the heart ventricle. A first rod portion 958 of the rod 950 proximal of the bend 956, such as the portion closer to the circumferentially disposed portion 902, and a second rod portion 960 of the rod 950 distal of the bend 956, such as the portion closer to the anchor 970, can have different orientations. In some instances, the bend 956 can be formed in the rod 950 during a portion of the systole phase. In some instances, the rod 950 can assume the bend during late systole. In some instances, the first rod portion 958 can assume a perpendicularly or substantially perpendicularly orientation, while the second rod portion 960 assumes an oblique orientation, relative to the plane of the circumferentially disposed portion 902. In some instances, both the first and second rod portions 958, 960 can have oblique orientations relative to the plane of the circumferentially disposed portion 902 during at least a portion of the systole phase of the heart ventricle, such as late systole.

The circumferentially disposed portion 902 and/or rod 950 can remain rotated relative to the rod 950 while the rod 950 forms the bend 956. In some instances, at least a portion of the circumferentially disposed portion 902 and/or rod 950 can rotate further around the same direction as the ventricle contracts further. In some instances, the plurality of coil springs 920 can be compressed to accommodate contraction of the heart ventricle. In some instances, the plurality of coil springs 920 can be further compressed, such as to a second compressed state, relative to that during early systole.

In some instances, as the heart ventricle transitions from systole to diastole, the circumferentially disposed portion 902 can assume the unrotated position. For example, the plurality of torsion springs 930 and/or central torsion spring can transform back to the relaxed state. The rod 950 can straighten to assume the unbent configuration. In some instances, the plurality of coiled springs 920 can transform back to the relaxed state.

It will be understood that although rotation of the circumferentially disposed portion 902, compression and/or relaxation of the coil springs 920, and bending and/or straightening of the rod 950, are described as comprising various states, the transformations can be continuous to accommodate contraction and relaxation of the heart ventricle. The springs as described herein can comprise a respective spring constant selected to provide desired expansion, while accommodating compression of the heart. In some instances, a spring as described herein can comprise a variable spring constant, for example to reduce resistance to compression and provide desired expansion at target lengths. In some instances, a spring can comprise a plurality of springs in series and/or in parallel. In some instances, a spring can comprise an inner spring rod, for example comprising a sheathed spring element. The springs can comprise any number of different materials, including polymers, nitinol, and/or steel.

In some instances, one or more of the medical implant devices 600, 700, 800, 900 described with reference to FIGS. 6, 7, 8 and 9 can be used in combination with one or more of the medical implant assemblies 200, 400 described with reference to FIGS. 2, 3, and 4.

### Additional Description of Examples

Provided below is a list of examples, each of which may include aspects of any of the other examples disclosed herein. Furthermore, aspects of any example described above may be implemented in any of the numbered examples provided below.

Example 1: A medical implant assembly comprising a longitudinally expandable member comprising an elongate portion configured to be reversibly expandable along a longitudinal dimension, the longitudinally expandable member being configured to be: secured to a ventricular heart wall surface oriented toward a heart ventricle, expanded to an expanded state as the heart ventricle expands during diastole, and compressed to a compressed state as the heart ventricle contracts during systole. The assembly can include a first anchor configured to be coupled to a first portion of the longitudinally expandable member to secure the first portion of the longitudinally expandable member to a first location on the ventricular heart wall surface, and a second anchor configured to be coupled to a second portion of the longitudinally expandable member to secure the second portion of the longitudinally expandable member to a second location on the ventricular heart wall surface.

Example 2: The assembly of any example herein, in particular example 1, wherein the longitudinally expandable member comprises a length of between 3 centimeters (cm) and 10 centimeters (cm).

Example 3: The assembly of any example herein, in particular example 1 or 2, wherein the longitudinally expandable member comprises a length of between 25% to 75% of a longitudinal dimension of the heart ventricle.

Example 4: The assembly of any example herein, in particular example 3, wherein the heart ventricle is a left heart ventricle.

Example 5: The assembly of any example herein, in particular examples 1 to 4, wherein the longitudinally expandable member comprises a first opening at the first portion configured to receive at least a portion of the first anchor, and a second opening at the second portion configured to receive at least a portion of the second anchor.

Example 6: The assembly of any example herein, in particular examples 1 to 5, wherein the longitudinally expandable member comprises a linear wave spring, a first surface of the linear wave spring being configured to be oriented toward the ventricular heart wall surface and a second surface of the linear wave spring being configured to be oriented away from the ventricular heart wall surface.

Example 7: The assembly of any example herein, in particular examples 1 to 6, wherein the first anchor comprises a first spiral portion configured to be screwed into the ventricular heart wall surface, and the second anchor comprises a second spiral portion configured to be screwed into the ventricular heart wall surface.

Example 8: The assembly of any example herein, in particular examples 1 to 7, wherein the longitudinally expandable member comprises a shape memory material.

Example 9: The assembly of any example herein, in particular examples 1 to 7, wherein the longitudinally expandable member comprises an electrically activated polymer and wherein the assembly further comprises: a generator configured to generate an electrical signal to activate the electrically activated polymer; a controller comprising a cardiac cycle monitor configured to monitor the cardiac cycle and configured to trigger the generator to generate the electrical signal in response to the heart ventricle being in diastole; a wire coupled to the generator and comprising at least a portion configured to be disposed within the heart ventricle to be coupled to the longitudinally expandable member; and an electrode coupled to the wire and configured to be disposed within the heart ventricle and in contact with the longitudinally expandable member to deliver the electrical signal for electrically activating the longitudinally expandable member.

Example 10: The assembly of any example herein, in particular examples 1 to 9, wherein the longitudinally expandable member is configured to be secured to the ventricular heart wall surface along an inner subendocardial fiber axis.

Example 11: The assembly of any example herein, in particular examples 1 to 10, further comprising: a plurality of longitudinally expandable members configured to be secured to respective positions on the ventricular heart wall surface and aligned along an inner subendocardial fiber axis; and a pair of anchors configured to be coupled to a respective one of the plurality of longitudinally expandable members to secure the respective one of the plurality of longitudinally expandable member to the ventricular heart wall surface.

Example 12: A delivery assembly comprising a proximal handle, and an elongate shaft extending distally from the proximal handle, where the elongate shaft can include a first delivery lumen and a second delivery lumen. The first delivery lumen can extend along a longitudinal axis of the elongate shaft and configured to receive a longitudinally expandable member, the longitudinally expandable member comprising an elongate portion configured to be reversibly expandable along a longitudinal dimension. The second delivery lumen can extend along the longitudinal axis and configured to receive an anchor configured to secure the longitudinally expandable member to a ventricular heart wall surface oriented toward a heart ventricle.

Example 13: The assembly of any example herein, in particular example 12, wherein the first delivery lumen is disposed around the second delivery lumen.

Example 14: The assembly of any example herein, in particular example 13, wherein the first delivery lumen is configured to receive the longitudinally expandable member in a helical configuration.

Example 15: The assembly of any example herein, in particular example 13, wherein the first delivery lumen is configured to receive a plurality of longitudinally expandable members arranged in a helical configuration.

Example 16: The assembly of any example herein, in particular example 12, wherein the first delivery lumen is disposed adjacent to the second delivery lumen.

Example 17: The assembly of any example herein, in particular examples 12 to 16, wherein the assembly is configured to position a first portion of the longitudinally expandable member at a first position on the ventricular heart wall surface; advance the anchor through a first portion of the longitudinally expandable member and into a first location of the heart ventricular wall surface; and advance a second anchor through a second portion of the longitudinally expandable member and into a second location of the heart ventricular wall surface.

Example 18: The assembly of any example herein, in particular example 17, wherein the assembly is configured to screw the anchor and the second anchor through the first portion and the second portion of the longitudinally expandable member and into the heart ventricular wall surface.

Example 19: The assembly of any example herein, in particular examples 12 to 18, further comprising a rod comprising a distal end portion configured to engage with the anchor, the rod being configured to be advanced the anchor through the second delivery lumen and screw the second anchor through the first portion of the longitudinally expandable member.

Example 20: The assembly of any example herein, in particular examples 12 to 19, wherein the second delivery lumen is configured to receive a plurality of anchors preloaded therein,

Example 21: A medical implant device comprising a circumferentially disposed portion configured to reversibly expand radially, and a plurality of anchors configured to be coupled to the circumferentially disposed portion to couple the circumferentially disposed portion to a circumferential portion of a ventricular heart wall surface. The circumferentially disposed portion can be configured to be secured to the circumferential portion of a heart ventricle wall surface oriented toward a heart ventricle, and to assume a radially expanded state during at least a portion of a diastole phase of the heart ventricle, and assume a radially collapsed state during at least a portion of a systole phase of the heart ventricle.

Example 22: The device of any example herein, in particular example 21, wherein the circumferentially disposed portion is configured to be secured to a circumferential portion of the heart ventricle wall surface aligned with a subendocardial circumferential fiber.

Example 23: The device of any example herein, in particular example 21, wherein the circumferentially disposed portion is configured to be secured to a circumferential portion of the heart ventricle wall surface at an infra-annular location.

Example 24: The device of any example herein, in particular example 21, wherein the circumferentially disposed portion is configured to be secured to a circumferential portion of the heart ventricle wall surface at an apical location.

Example 25: The device of any example herein, in particular example 24, wherein the circumferentially disposed portion is configured to be secured to a circumferential portion of the heart ventricle wall surface inferior of and adjacent to papillary muscles of the heart ventricle.

Example 26: The device of any example herein, in particular examples 21 to 25, wherein the circumferentially disposed portion comprises a coil spring.

Example 27: The device of any example herein, in particular example 26, wherein the coil spring is a ring-shaped coil spring.

Example 28: The device of any example herein, in particular example 27, further comprising a partial ring-shaped member comprising a recess configured to receive the coil spring, the partial ring-shaped member being configured to be secured to the heart ventricle wall surface.

Example 29: The device of any example herein, in particular examples 21 to 25, wherein the circumferentially disposed portion comprises a linear wave spring, a first surface of the linear wave spring being configured to be oriented toward the ventricular heart wall surface and a second surface of the linear wave spring being configured to be oriented away from the ventricular heart wall surface.

Example 30: The device of any example herein, in particular example 29, wherein the linear wave spring forms a partial ring.

Example 31: The device of any example herein, in particular examples 21 to 25, wherein the circumferentially disposed portion comprises a plurality of radially extending spokes, a first end portion of each of the plurality of radially extending spokes being configured to be oriented toward a center of the circumferentially disposed portion and a second end portion of each of the plurality of radially extending spokes being configured to be configured to be oriented toward an edge of the circumferentially disposed portion. The circumferentially disposed portion can include a plurality of torsion springs, each of the plurality of torsion springs having a first end portion configured to be coupled to the second end portion of a respective one of the plurality of radially extending spokes, and a second end portion configured to be coupled to a respective portion of the ventricular heart wall surface.

Example 32: The device of any example herein, in particular example 31, where the circumferentially disposed portion further comprises a central torsion spring, the central torsion spring comprising a respective end portion being coupled to each of the plurality of radially extending spokes.

Example 33: The device of any example herein, in particular example 31 or 32, wherein a radially extending spoke comprises a linear coil spring along at least a portion of a length of the radially extending spoke.

Example 34: The device of any example herein, in particular example 33, wherein each of the plurality radially extending spokes comprises a linear coil spring extending along at least a portion of a length of the radially extending spoke.

Example 35: The device of any example herein, in particular examples 31 to 34, further comprising a rod comprising a first end portion coupled to a central portion of the circumferentially disposed portion, and an anchor coupled to a second end portion of the rod, the anchor being configured to be coupled to an apical portion of the heart ventricle wall surface.

Example 36: The device of any example herein, in particular example 35, wherein at least a portion of the rod is configured to be oriented perpendicularly relative to a plane of the circumferentially disposed portion during at least a portion of the diastole phase of the heart ventricle, and oriented obliquely relative to the plane of the circumferentially disposed portion during at least a portion of the systole phase of the heart ventricle.

Example 37: The device of any example herein, in particular example 36, wherein the rod is configured to bend during at least a portion of the systole phase of the heart ventricle.

Example 38: The device of any example herein, in particular examples 35 to 37, wherein the anchor comprises a spiral anchor configured to be screwed into the heart ventricle wall surface.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other embodiment(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an clement, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A method for assisting a heart ventricle, the method comprising:
providing a longitudinally expandable member that is reversibly expandable along a longitudinal dimension, wherein the longitudinally expandable member comprises adjacent abutting portions meeting at respective bends that form acute angles along the longitudinal dimension;
securing a first end portion of the longitudinally expandable member to a first wall portion of the heart ventricle via a first anchor engagement feature associated with the first end portion; and
securing a second end portion of the longitudinally expandable member to a second wall portion of the heart ventricle via a second anchor engagement feature associated with the second end portion, the respective bends between the first and second end portions allowing augmenting expansion of the heart ventricle using spring force of the longitudinally expandable member to thereby improve heart function.

2. The method of claim 1, wherein providing the longitudinally expandable member comprises providing a longitudinally expandable member having acute angles of the respective ends decrease with compression of the longitudinally expandable member and increase with expansion of the longitudinally expandable member.

3. The method of claim 1, wherein:
providing the longitudinally expandable member comprises providing a longitudinally expandable member comprising a linear wave spring configuration; and
securing the first end portion of the longitudinally expandable member to the first wall portion and the second end portion of the longitudinally expandable member to the second wall portion comprises orienting a first surface of the longitudinally expandable member toward a respective wall portion of the heart ventricle and orienting a second opposing surface of the longitudinally expandable member away from the respective wall portion of the heart ventricle.

4. The method of claim 1, wherein:
securing the first end portion of the longitudinally expandable member to the first wall portion comprises inserting a first anchor through a first opening extending through the first end portion to sandwich a first portion of the longitudinally expandable member between the first anchor and the first wall portion of the heart ventricle; and
securing the second portion of the longitudinally expandable member to the second wall portion comprises inserting a second anchor through a second opening extending through the second end portion to sandwich a second portion of the longitudinally expandable member between the second anchor and the second wall portion of the heart ventricle.

5. The method of claim 1, wherein providing the longitudinally expandable member comprises providing a longitudinally expandable member comprising a length of between 3 centimeters (cm) and 10 centimeters (cm).

6. The method of claim 1, wherein providing the longitudinally expandable member comprises providing a longitudinally expandable member comprising a length of between 25% to 75% of a longitudinal dimension of the heart ventricle.

7. The method of claim 1, wherein securing the first end portion of the longitudinally expandable member to the first wall portion and securing the second end portion of the longitudinally expandable member to the second wall portion comprise securing the longitudinally expandable member to the first and second wall portions while the longitudinally expandable member is in a compressed state to bias the longitudinally expandable member for lengthening a ventricular heart wall and augmenting expansion of the heart ventricle.

8. A method for assisting a heart ventricle, the method comprising:
providing a longitudinally expandable member comprising a reversibly foldable configuration;
securing a first end portion of the longitudinally expandable member to a first portion of a ventricular heart wall; and
securing a second end portion of the longitudinally expandable member to a second portion of the ventricular heart wall for allowing augmenting expansion of the heart ventricle using spring force of the longitudinally expandable member to thereby improve heart function.

9. The method of claim 8, wherein providing the longitudinally expandable member comprises providing a longitudinally expandable member comprising an elongate portion adapted to fold and assume a compressed configuration along a longitudinal dimension, and unfold and assume an expanded configuration along the longitudinal dimension.

10. The method of claim 8, wherein providing the longitudinally expandable member comprises providing a longitudinally expandable member comprising an accordion configuration.

11. A method of assisting a heart ventricle, the method comprising:
advancing a delivery assembly to the heart ventricle, the delivery assembly holding a longitudinally expandable member comprising a plurality of folds that reversibly expand along a longitudinal dimension of the longitudinally expandable member;
deploying the longitudinally expandable member from the delivery assembly for securing the longitudinally expandable member to a ventricular heart wall;
orienting a first surface of the longitudinally expandable member toward the ventricular heart wall and a second opposing surface of the longitudinally expandable member away from the ventricular heart wall;
coupling a first anchor to a first end portion of the longitudinally expandable member to secure the first end portion of the longitudinally expandable member to a first location on the ventricular heart wall; and
coupling a second anchor to a second end portion of the longitudinally expandable member to secure the second end portion of the longitudinally expandable member to a second location on the ventricular heart wall, thereby allowing augmenting expansion of the heart ventricle using spring force of the longitudinally expandable member and improvement of heart function.

12. The method of claim 11, wherein advancing the delivery assembly to the heart ventricle holding the longitudinally expandable member comprises advancing the delivery assembly to the heart ventricle holding a longitudinally expandable member comprising a first opening extending through the first end portion, and a second opening extending through the second end portion.

13. The method of claim 12, wherein coupling the first anchor comprises screwing a spiral portion of the first anchor into the ventricular heart wall through the first opening of the longitudinally expandable member, and the coupling the second anchor comprises screwing a spiral portion of the second anchor into the ventricular heart wall through the second opening of the longitudinally expandable member.

14. The method of claim 11, wherein coupling the first anchor and coupling the second anchor comprises sandwiching a first respective portion of the longitudinally expandable member between a proximal portion of the first anchor and the ventricular heart wall, and a second respective portion of the longitudinally expandable member between a proximal portion of the second anchor and the ventricular heart wall such that the first surface is oriented toward the ventricular heart wall and the second surface is oriented away from the ventricular heart wall.

15. The method of claim 11, wherein advancing the delivery assembly to the heart ventricle holding the longitudinally expandable member comprises advancing the delivery assembly to the heart ventricle holding a longitudinally expandable member comprising a linear wave spring, a first surface of the linear wave spring being adapted to be oriented toward the ventricular heart wall and a second surface of the linear wave spring being adapted to be oriented away from the ventricular heart wall.

16. The method of claim 11, wherein advancing the delivery assembly to the heart ventricle holding the longitudinally expandable member comprises advancing the delivery assembly to the heart ventricle holding a longitudinally expandable member comprising an electrically activated polymer and wherein the method further comprises:
providing a generator adapted to generate an electrical signal to activate the electrically activated polymer;
providing a controller comprising a cardiac cycle monitor adapted to monitor the cardiac cycle and adapted to trigger the generator to generate the electrical signal in response to the heart ventricle being in diastole; and
disposing an electrode within the heart ventricle and in contact with the longitudinally expandable member to deliver the electrical signal for electrically activating the longitudinally expandable member, the electrode being coupled to a wire extending from the generator.

17. The method of claim 11, wherein deploying the longitudinally expandable member from the delivery assembly comprises aligning the longitudinally expandable member with an inner subendocardial fiber axis.

18. The method of claim 11, further comprising:
deploying a plurality of longitudinally expandable members from the delivery assembly and aligning each of the plurality of longitudinally expandable members along an inner subendocardial fiber axis; and
coupling a pair of anchors to a respective one of the plurality of longitudinally expandable members to secure the respective one of the plurality of longitudinally expandable members to the ventricular heart wall.

19. The method of claim 11, wherein advancing the delivery assembly to the heart ventricle comprises advancing a delivery assembly comprising:
a proximal handle; and
an elongate shaft extending distally from the proximal handle, the elongate shaft comprising:
a first delivery lumen extending along a longitudinal axis of the elongate shaft and configured to receive a longitudinally expandable member, and
a second delivery lumen extending along the longitudinal axis and configured to receive an anchor configured to secure the longitudinally expandable member to a ventricular heart wall surface oriented toward a heart ventricle.

20. The method of claim 19, wherein advancing the delivery assembly to the heart ventricle comprises advancing a delivery assembly comprising an elongate shaft having a first delivery lumen that is disposed around the second delivery lumen.
